(19) United States
(12) Patent Application Publication
(10) Pub. No.: US 2025/0018108 A1
(43) Pub. Date: Jan. 16, 2025

(54) CANNULA, INFUSION DEVICES AND METHODS

(71) Applicant: CONVATEC LIMITED, Flintshire (GB)

(72) Inventors: Omid Mostafaei, Lejre (DK); Jens Schultz, Lejre (DK)

(21) Appl. No.: 18/902,772

(22) Filed: Sep. 30, 2024

Related U.S. Application Data
(63) Continuation of application No. PCT/GB2023/050819, filed on Mar. 29, 2023.

(30) Foreign Application Priority Data
Apr. 8, 2022 (GB) GB2205204.7

(51) Int. Cl. A61M 5/142

(52) U.S. Cl. CPC A61M 5/14248; A61M 2202/0007; A61M 2202/04; A61M 2205/0216; A61M 2205/0255; A61M 2205/10; A61M 2209/088; A61M 2210/04

(57) ABSTRACT

A cannula for subcutaneous infusion of a therapeutic agent, an infusion device for subcutaneous delivery of a therapeutic agent to a patient comprising the cannula, and a method of administering a therapeutic agent via an infusion device, the cannula comprising a tubular body member comprising a tubular wall at least partly enclosing a longitudinal extending internal bore, a distal portion of the tubular body member having a distal tip end, wherein the distal portion comprises a weakened section comprising a first plurality of holes and a second plurality of holes, the weakened section being capable of allowing the cannula to flex in an area comprising the weakened section when the cannula is exposed to a compression force and/or an increased internal pressure, and wherein the first plurality of holes comprise holes of larger diameter than the second plurality of holes.

### TECHNICAL FIELD

This disclosure describes infusion and cannula devices and methods for use of the infusion and cannula devices. The medical devices disclosed herein are configured for introduction of a therapeutic agent (e.g., a drug), including a therapeutic liquid or suspension or other suitable material, into a subject. In certain embodiments, the medical device is an infusion device comprising a cannula configured for this purpose.

### BACKGROUND

Infusion devices comprising a flexible cannula, are designed to be inserted into the skin by means of an introducer needle and the needle is then removed. A therapeutic agent, such as insulin, is then delivered through the cannula. However, there is a risk that the cannula may become occluded (e.g., obstructed) upon delivery of the therapeutic agent. This can happen when the tip of the cannula is blocked for instance by inflammation in the tissue, or due to kinking of the cannula.

It is therefore desirable to minimize the risk of blocking of the cannula outlet, and/or the risk that kinking obstructs the discharge of the therapeutic agent (e.g., a drug). Previous attempts to circumvent these obstructions, including the ues of cannulas with several openings (see, e.g., US Patent Pre-Grant Publication No. 2013/0245555) have been reported, but the risk of kinking and occlusion (e.g., obstruction) still remains. WO2017/125817 discloses various improvements to the configurations of cannula and infusion devices that provide reduced risk of blocking of the cannula outlet, and/or the risk that kinking obstructs the discharge of the therapeutic agent (e.g., a drug).

It is an object of embodiments of the invention to provide additional improvements and/or at least mitigate one or more problems associated with the known arrangements.

### SUMMARY OF THE INVENTION

According to an aspect of the invention, there is provided, a cannula for subcutaneous infusion of a therapeutic agent, the cannula comprising a tubular body member comprising a tubular wall at least partly enclosing a longitudinal extending internal bore, a distal portion of the tubular body member having a distal tip end, wherein the distal portion comprises a weakened section comprising a first plurality of holes and a second plurality of holes, the weakened section being capable of allowing the cannula to flex in an area comprising the weakened section when the cannula is exposed to a compression force and/or an increased internal pressure, and wherein the first plurality of holes comprise holes of larger diameter than the second plurality of holes.

The distal tip end may comprise at least one tip opening.

The first plurality of holes may be closer to the distal tip end than the second plurality of holes.

According to another aspect of the invention, there is provided a cannula for subcutaneous infusion of a therapeutic agent. The cannula may include a tubular body member comprising a tubular wall at least partly enclosing a longitudinal extending internal bore, a distal portion of the tubular body member having a distal tip end including at least one tip opening, wherein the distal portion includes a weakened section. The weakened section may include a first plurality of holes and a second plurality of holes. The first plurality of holes may be closer to the distal end tip than the second plurality of holes. The weakened section may be capable of allowing the cannula to flex in an area including the weakened section when the cannula is exposed to a compression force and/or an increased internal pressure. The first plurality of holes may include holes of a larger diameter than the second plurality of holes.

For the avoidance of doubt, any of the features described herein apply equally to any aspect of the invention. For example, the cannula may include any one or more features of the infusion device relevant to the cannula and/or the method may include any one or more features or steps relevant to one or more features of the cannula or the infusion device.

According to another aspect of the invention, there is provided a cannula for subcutaneous infusion of a therapeutic agent, the cannula including a tubular body member having a tubular wall at least partly enclosing a longitudinal extending internal bore, a distal portion of the tubular body member having a distal tip end including at least one tip opening, wherein the distal portion has a weakened section including a first plurality of holes and a second plurality of holes, the first plurality of holes being closer to the distal end tip than the second plurality of holes, the weakened section being capable of allowing the cannula to flex in an area including the weakened section when the cannula is exposed to a compression force and/or an increased internal pressure, and wherein the first plurality of holes include holes of a larger diameter than the second plurality of holes.

Beneficially, the inclusion of holes of a larger diameter closer to the distal end tip enables the delivery of therapeutic agents closer to the tip. Furthermore, in the event that the cannula becomes kinked or bent, the kink or bend is more likely to occur in the region having holes of a larger diameter (i.e. the first plurality of holes), thereby leaving the holes of the second plurality of holes fully open so that therapeutic agents can continue to be delivered from the cannula.

The number of holes in the first plurality of holes may be between 4 and 16. Each hole of the first plurality of holes may have a diameter in the range of 61 to 75 pm. Each of the first plurality of holes may be at the same distance from the tip opening.

The number of holes in the second plurality of holes may be between 4 and 16. Each hole of the second plurality of holes may have a diameter in the range of 50 to 60 pm.

The weakened section may include a third plurality of holes. The number of holes in the third plurality of holes may be between 4 and 16. Each hole of the third plurality of holes may have a diameter in the range of 50 to 55 pm. The third plurality of holes may be further from the distal end tip than the second plurality of holes.

The distal tip may be shaped to a point. Beneficially, the shaped distal tip minimises the insertion impact of the cannula.

A length of the distal end of the cannula may be less than 3.5 mm. Additionally, or alternatively, an outer diameter of the distal end may be less than 1.5 mm.

The distal end may further include a corrugated portion. The distal portion of the cannula may include PTFE (polytetrafluoroethylene), FEP (fluorinated ethylene propylene), rubber, PE (polyethylene) material or silicone base materials.

The cannula may be arranged so that at least a portion of the therapeutic agent is released from the distal tip end. The cannula may be exposed to a compression force and/or an increased internal pressure. The internal pressure in the longitudinal extending bore may exceed the internal pressure at the tip opening. The weakened section may provide a fluid communication between the internal bore and the outside of the cannula. The cannula may be configured to be insertable with an insertion needle. The cannula may be configured such that the weakened section is below a basal membrane of the skin when the cannula is subcutaneously placed.

The weakened section may have a compression strength smaller than the compression strength of the remaining portions of the tubular body member.

According to another aspect of the invention, there is provided an infusion device for subcutaneous delivery of a therapeutic agent to a patient comprising: a cannula comprising a tubular body member comprising a tubular wall at least partly enclosing a longitudinal extending internal bore, a distal portion of the tubular body member having a distal tip end, wherein the distal portion comprises a weakened section comprising a first plurality of holes and a second plurality of holes, the weakened section being capable of allowing the cannula to flex in an area comprising the weakened section when the cannula is exposed to a compression force and/or an increased internal pressure, wherein the first plurality of holes comprise holes of larger diameter than the second plurality of holes; and a hub part configured to be fastened onto a patient's skin via a mounting pad.

The distal tip end may comprise at least one tip opening.

The first plurality of holes may be closer to the distal tip end than the second plurality of holes.

According to another aspect of the invention there is provided an infusion device for subcutaneous delivery of a therapeutic agent to a patient including: a cannula including a tubular body member including a tubular wall at least partly enclosing a longitudinal extending internal bore, a distal portion of the tubular body member having a distal tip end including at least one tip opening, wherein the distal portion includes a weakened section including a first plurality of holes and a second plurality of holes, the first plurality of holes closer to the distal end tip than the second plurality of holes, the weakened section being capable of allowing the cannula to flex in an area including the weakened section when the cannula is exposed to a compression force and/or an increased internal pressure, wherein the first plurality of holes include holes of larger diameter than the second plurality of holes; and a hub part configured to be fastened onto the patient's skin via a mounting pad.

The device may be configured such that at least a portion of the therapeutic agent is released from the distal tip end of the cannula.

The weakened section may have a compression strength smaller than the compression strength of the remaining portions of the tubular body member.

When the cannula is exposed to a compression force and/or an increased internal pressure and the internal pressure in the longitudinal extending bore exceeds the internal pressure that the tip opening, the weakened section may provide a fluid communication between the internal bore and the outside of the cannula.

The distal end of the cannula may include a corrugated portion. The distal portion of the cannula may include PTFE (polytetrafluoroethylene), FEP (fluorinated ethylene propylene), rubber, PE (polyethylene) material or silicone base materials.

The cannula may be configured to be insertable with an insertion needle.

The cannula may be configured such that the weakened section is positioned below a basal membrane of the skin when the cannula is subcutaneously placed.

A length of the distal end of the cannula may be less than 3.5 mm, and/or an outer diameter of the distal end may be less than 1.5 mm.

Each hole of the first plurality of holes may have a diameter in the range of 61 to 75 pm. Each hole of the second plurality of holes may have a diameter in the range of 50 to 60 pm.

The device may further include a pump in fluid connection with a reservoir configured to store medication/drugs.

The subcutaneously placed distal portion of said cannula may include a soft material such as PTFE (polytetrafluoroethylene), FEP (fluorinated ethylene propylene), rubber, PE (polyethylene) material or silicone base materials.

The infusion device may be configured for subcutaneous infusion of one or more therapeutic agents. The therapeutic agent may include insulin.

According to another embodiment of the invention, there is provided a method of administering a therapeutic agent via an infusion device, the method comprising: providing a cannula comprising a tubular body member comprising a tubular wall at least partly enclosing a longitudinal extending internal bore, a distal portion of the tubular body member having a distal tip end comprising at least one tip opening, wherein the tubular wall comprises a weakened section comprising a first plurality of holes and a second plurality of holes, and wherein the first plurality of holes comprise holes of larger diameter than the second plurality of holes; and causing the cannula to flex in an area comprising the weakened section when 1) the cannula is exposed to a compression force whereby the tip opening is substantially closed and/or 2) the cannula is exposed to an increased internal pressure exceeding the pressure at the tip opening, wherein the compression force or increased internal pressure opens the weakened section allowing fluid communication between the internal bore and the external environment, thereby discharging the therapeutic agent.

The first plurality of holes may be closer to the distal tip end than the second plurality of holes.

According to another embodiment of the invention there is provided a method of administering a therapeutic agent via an infusion device, the method including: providing a cannula including a tubular body member including a tubular wall at least partly enclosing a longitudinal extending internal bore, a distal portion of the tubular body member having a distal tip end including at least one tip opening, wherein the tubular wall includes a weakened section including a first plurality of holes and a second plurality of holes, the first plurality of holes being closer to the distal end tip than the second plurality of holes, and wherein the first plurality of holes include holes of larger diameter than the second plurality of holes; and causing the cannula to flex in an area including the weakened section when 1) the cannula is exposed to a compression force whereby the tip opening is substantially closed and/or 2) the cannula is exposed to an increased internal pressure exceeding the pressure at the tip opening, wherein the compression force or increased internal pressure opens the weakened section allowing fluid communication between the internal bore and the external environment, thereby discharging the therapeutic agent.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

Embodiments of the invention will now be described, by way of example only, with reference to the accompanying figures, in which:

FIG. 1 shows an example of a known infusion device;

FIG. 2A is a planar view of an example of a known cannula;

FIG. 2B is another planar view of the cannula shown in FIG. 2A;

FIG. 3 is an example of a cross-sectional view of the cannula shown in FIG. 2B;

FIG. 4 shows a cannula according to the present invention;

FIG. 5 is a planar view of an embodiment of a cannula according to the present invention;

FIG. 6 is a planar view of an alternative embodiment of a cannula according to the present invention; and

FIG. 7 is a cross-sectional view of the cannula of FIG. 6.

### DETAILED DESCRIPTION OF EMBODIMENTS OF THE INVENTION

Examples of the invention are now described with reference to the accompanying drawings.

This disclosure describes infusion devices that, in some cases, include: a cannula having a tubular body member with a proximal portion and a distal portion subcutaneously placed when the infusion device is placed on an outside surface of a patients skin, the tubular body member comprising a tubular wall enclosing a longitudinal extending internal bore, the distal portion having a distal end with a tip end comprising at least one tip opening, allowing a portion of a therapeutic agent (e.g., a drug) conveyed through the internal bore in the tubular body member to discharge; and a hub part configured to be fastened onto the patient's skin via a mounting pad.

This disclosure also describes cannulas that, in some cases, reduce or minimize kinking and/or occlusion of the subcutaneously placed devices.

In some embodiments, the cannula comprises a weakened section comprising a first plurality of holes and a second plurality of holes, the first plurality of holes closer to the distal end tip than the second plurality of holes, and wherein the first plurality of holes comprise holes of a larger diameter than the second plurality of holes. In other embodiments, the cannula is adapted to flex in an area comprising the weakened section when the cannula is exposed to a compression force and/or an increased therapeutic agent or internal pressure taking place inside the longitudinal extending bore, wherein the therapeutic agent or internal pressure exceeds the pressure at the tip opening, whereby the first plurality of holes and the second plurality of holes of the weakened section provides fluid communication between the internal bore and the outside of the cannula.

The expression “an area comprising the weakened section” denotes that part of the cannula wall comprising the weakened section or/and the neighbouring wall of a weakened section in circumferential direction of the cannula.

A “weakened section” includes a part of the wall that has a smaller compression strength and/or requires a smaller force for elongation than the rest of the cannula wall. The weakened section of the wall may be between 0.1 pm to 0.5 mm thinner than the surrounding wall. The weakened section may also be the result of specific materials at the site of the weakened section, and thus has a smaller compression and elongation force than the rest of the cannula. In some embodiments, the materials in the weakened section may comprise PTFE (polytetrafluoroethylene), rubber or PE (polyethylene).

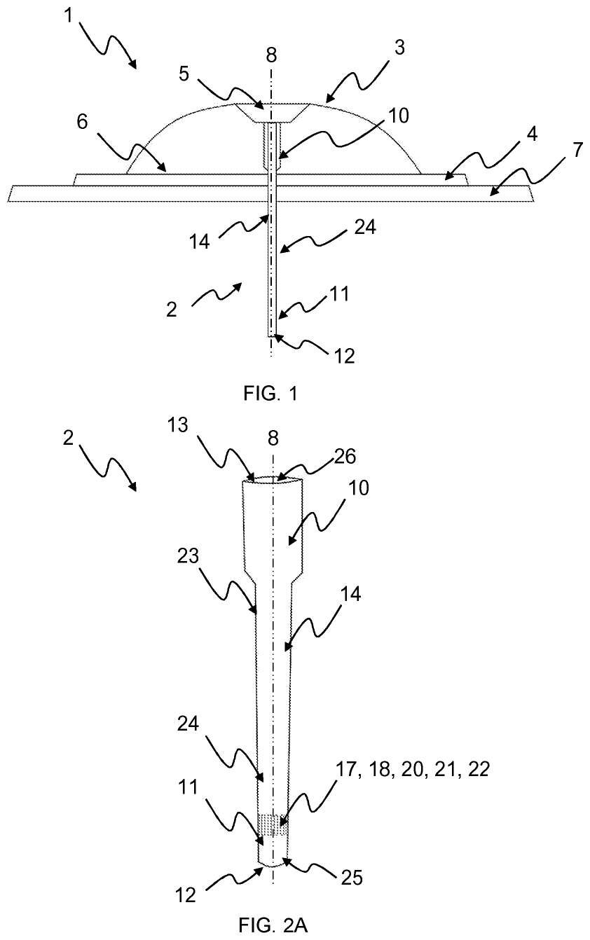

FIG. 1 illustrates schematically an example of an infusion device 1. The infusion device 1 includes a cannula 2 having a tubular body member 14 with a proximal portion 10 and a subcutaneously placed distal portion 24 having a distal end 11. The distal portion 24 of the cannula 2 is provided with a tip opening 12 allowing a portion of a drug conveyed through the tubular body member 14 to discharge.

The infusion device according to FIG. 1 further includes a hub part 3 configured to be fastened onto an outside of the surface of a patient's skin 7 via a mounting pad 4. The mounting pad 4 may be provided with an adhesive layer, configured to adhere to a patient's skin, and a not shown removable release liner that covers the adhesive layer. The mounting pad 4 may be a dressing, a plaster, an adhesive pad or the like, and the mounting pad may be configured in various shapes such as oval, circular, triangular rectangular etc. According to some implementations, the infusion device 1 may include the hub part 3 having a main plane being essentially parallel to the skin of the patient, when the infusion set is attached to a patient. The hub part 3 may have a main surface 6 being closest to the skin of a patient, and the main surface 6 may be provided with fastening means such as the mounting pad 4 for fastening the infusion device to the skin 7 of the patient.

In the infusion device according to FIG. 1, the cannula 2 extends from the hub part 3 through the fastening means or mounting pad 4. Alternatively, the cannula 2 may extend from the hub part 3 of the infusion device essentially along an inclined axis of insertion (not shown). As shown in FIG. 1, the hub part 3 may include a septum or barrier 5.

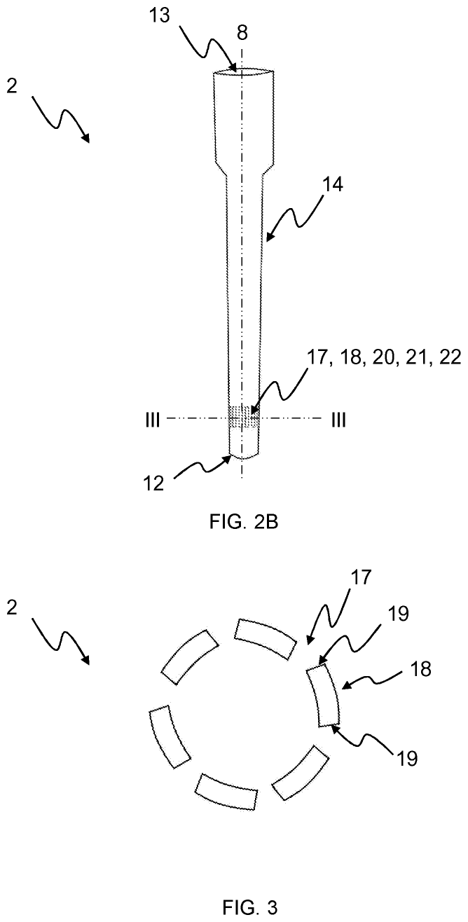

An example of a known cannula is illustrated in FIG. 2A. The cannula 2 is provided with a tubular body member 14 having a longitudinal extending internal bore 13—a conduit—extending along the longitudinal axis 8 of the cannula 2. A tubular wall 23 is surrounding the bore 13. (The conduit interconnects the infusion device with an inlet opening 26 in the top and a tip opening 12). The proximal portion 10 is configured for interconnection with the infusion device (not shown in FIG. 2A). The distal portion 24 is configured for subcutaneous placement by means of an insertion needle (not shown).

The tip opening or outlet 12 is arranged at the cannula tip or distal tip end 25 i.e., in the distal end 11 of the distal portion 24 of the cannula 2, opposite the interconnection with the infusion device. The outlet 12 may or may not be considered the primary outlet for drugs. In addition to the outlet 12, the cannula 2 is provided with weakened portions at least a first 21 and a second weakened 22 portion. In this embodiment, there is a number of weakened portions 20, each formed as slits 17 in the distal end 11 of the cannula. The slits (2-10 slits) are in the wall of the cannula 2 close to the tip 25 of the cannula 2 and all with substantially the same distance from the tip 25 and substantially with the same distance between the slits 17. The length of the slits is, in some cases, in a range of 0.2-1.5 mm, 0.4-0.8 mm, or 0.4-0.6 mm, and the distance of the weakened portion most distal to the tip of the cannula is in a range of 0.2-2.5 mm, 0.5-1.5 mm, or 0.75-1.25 mm and extends towards the proximal portion 10 of the cannula 2. The slits 17 are cut through the wall 23 allowing a fluid communication from the internal bore 13 to the outside of the cannula 2. The width of the slits 17 is, in some cases, in a range of 10-200 pm, 10-100 pm, or 10-50 pm. A laser may cut the slits 17.

The slits 17 may provide slats 18 between the slits 17. The slats 18 are flexible, and bend outwards when the cannula 2 is subjected to a compression force or an increased internal pressure. The sidewall of the slats 18 can be formed in different ways. One example is illustrated in FIG. 3 which is a cross-section of the cannula in FIG. 2B along the line Ill-Ill. Further, while FIGS. 2A and 2B show the slits 17 as narrow openings in the cannula 2, in some instances, each of the slits 17 is defined by a cut though the cannula 2 such that the slits 17 are normally in a closed or sealed configuration prior to flexing of the cannula and such that flexing of the cannula causes at least some of the slits 17 to open so as to allow the therapeutic agent to exit the cannula through the slits 17. In some instances, prior to flexing of the cannula, the slits 17 are closed or sealed such that the therapeutic agent does not exit through the slits.

The example shown in FIG. 3 shows a cannula 2 provided with six slits 17 thereby forming six slats 18 between them. The sidewalls 19 of the slats 18 are parallel to each other and parallel to the longitudinal axis 8 of the cannula 2 but all formed in such a way that the two side walls belonging to two opposing slats 18 form an opening/a slit 17 which is converging towards the internal bore 13. In some instances, the slits 17 are separated equidistantly from one another. In some instances, the distal end of the cannula 2 includes a tapered region where the tubular body member 14 forms the outlet 12. In some instances, the slits 17 are positioned on the cannula 2 across the intersection between the tapered region and a non-tapered region of the tubular body member 14. In some instances, the six slits 17 are 0.5 mm-0.8 mm in length along the tubular body member 14, and have a width of about 0.025 mm.

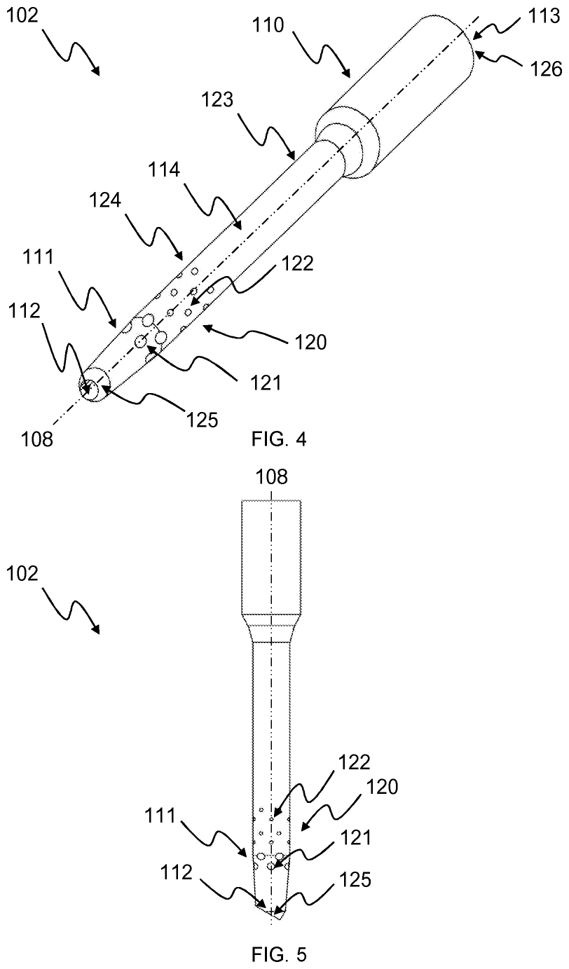

A cannula 102 according to an embodiment of the present invention will now be described with particular reference to FIGS. 4 and 5. The cannula 102 is applicable to the infusion device 1 according to some implementations. The cannula 102 is provided with a tubular body member 114 having a longitudinal extending internal bore or conduit 113 which extends along the longitudinal axis 108 of the cannula 102. A tubular wall 123 surrounds the bore 113. As described in relation to the cannula of FIG. 2, the conduit 113 interconnects the infusion device with an inlet opening 126 in the top and a tip opening 112. The cannula 102 includes a proximal portion 110, which is configured for interconnection with the infusion device (not shown in FIG. 4) as well as a distal portion 124 configured for subcutaneously placement by means of an insertion needle (not shown).

The outlet or tip opening 112 is arranged at the cannula tip or distal tip end 125 i.e. in the distal end 111 of the distal portion 124 of the cannula 102, opposite the interconnection with the infusion device. The distal tip end 125 may be shaped to a point, for example skived. The insertion impact of such a distal tip end 125 can, beneficially, be reduced in this way. The distal end may, in some embodiments of the invention, include a corrugated portion. The outlet 112 may or may not be considered the primary outlet for drugs. In addition to the outlet 112, the cannula 102 is provided with a weakened section 120. In this embodiment, the weakened section includes a first plurality of holes 121 and a second plurality of holes 122. The first plurality of holes 121 and the second plurality of holes 122 are formed in the wall 123 at the distal end 111 of the cannula 102.

The first plurality of holes 121, which may include 4 to 16 holes, for example, are positioned closer to the distal end tip 125 than the second plurality of holes 122, which may include 4 to 16 holes, for example.

In embodiments of the invention, the holes of the first plurality of holes 121 are at a distance in the range of 1.2 to 1.4 mm from the distal end tip 125. In embodiments of the invention, the holes of the first plurality of holes 121 are arranged in at least one row. Each row of the holes of the first plurality of holes may, for example, be equally spaced along the longitudinal axis 108 of the cannula 102. In embodiments of the invention, one row of the at least one row of the first plurality of holes 121 is at a distance of 1.2 mm from the distal end tip 125, a second row of the at least one row of the first plurality of holes 121 is at a distance of 1.3 mm from the distal end tip 125, and a third row of the at least one row of the first plurality of holes 121 is at a distance of 1.4 mm from the distal end tip 125.

In embodiments of the invention, the holes of the second plurality of holes 122 are at a distance in the range of 1.5 to 2.0 mm from the distal end tip 125. In embodiments of the invention, the holes of the second plurality of holes 122 are arranged in at least one row. Each row of the holes of the second plurality of holes 122 may, for example, be equally spaced along the longitudinal axis 108 of the cannula 102. In embodiments of the invention, one row of the at least one row of the second plurality of holes 122 is at a distance of 1.5 mm from the distal end tip 125, a second row of the at least one row of the second plurality of holes 122 is at a distance of 1.6 mm from the distal end tip 125, a third row of the at least one row of the second plurality of holes 122 is at a distance of 1.7 mm from the distal end tip 125, a fourth row of the at least one row of the second plurality of holes 122 is at a distance of 1.8 mm from the distal end tip 125, a fifth row of the at least one row of the second plurality of holes 122 is at a distance of 1.9 mm from the distal end tip 125, a sixth row of the at least one row of the second plurality of holes 122 is at a distance of 2.0 mm from the distal end tip 125.

In embodiments of the invention, each row of the first plurality of holes 121 and the second plurality of holes 122 may include, for example, 4 holes. Each of the 4 holes in each row of the first plurality of holes 121 and the second plurality of holes 122 may be equally spaced around the outer circumference of the tubular wall 123 such that each hole is spaced apart from its adjacent holes by 90°. Adjacent rows of the first plurality of holes 121 and the second plurality of holes 122 may be offset, for example by 90°. The adjacent rows of the first plurality of holes 121 and the second plurality of holes 122 may also be offset, for example by 90°. The arrangement of the first plurality of holes 121 and the second plurality of holes 122 advantageously enhances the mechanical integrity of the cannula 102.

In embodiments of the invention, the first plurality of holes 121 include holes having a larger diameter than the holes of the second plurality of holes 122. The holes of the first plurality of holes 121 may, for example, have a diameter in the range of 61 to 75 pm. The holes of the second plurality of holes 122 may, for example, have a diameter in the range of 50 to 60 pm. The provision of holes having a larger diameter closer to the distal tip end 125 advantageously ensures that more of the drug is delivered closer to the distal tip end 125. Furthermore, in the event that kinking of the cannula occurs, such kinking is more likely to occur in the region of the first plurality of holes 121 and delivery of the drug will still be possible via the numerous holes of the second plurality of holes 122. In embodiments of the invention, the total opening area of the first plurality of holes 121 and the second plurality of holes 122 may be approximately equal to the area of the tip opening 112.

The holes of each of the first plurality of holes 121 and the second plurality of holes 122 are cut through the wall 123 allowing a fluid communication from the internal bore 113 to the outside of the cannula 102. A laser may cut the holes of each of the first plurality of holes 121 and the second plurality of holes 122. Each hole of each of the first plurality of holes 121 and the second plurality of holes 122 has an opposing, or mirror, hole on the opposite side of the tubular wall 123. This advantageously reduces the time taken for the holes to be cut into the cannula 102.

When the cannula 102 is subjected to a compression force or an increased internal pressure such that the internal pressure in the longitudinal extending bore 113 exceeds the internal pressure at the tip opening 112, the weakened section 120 provides a fluid communication between the internal bore 113 and the outside of the cannula 102.

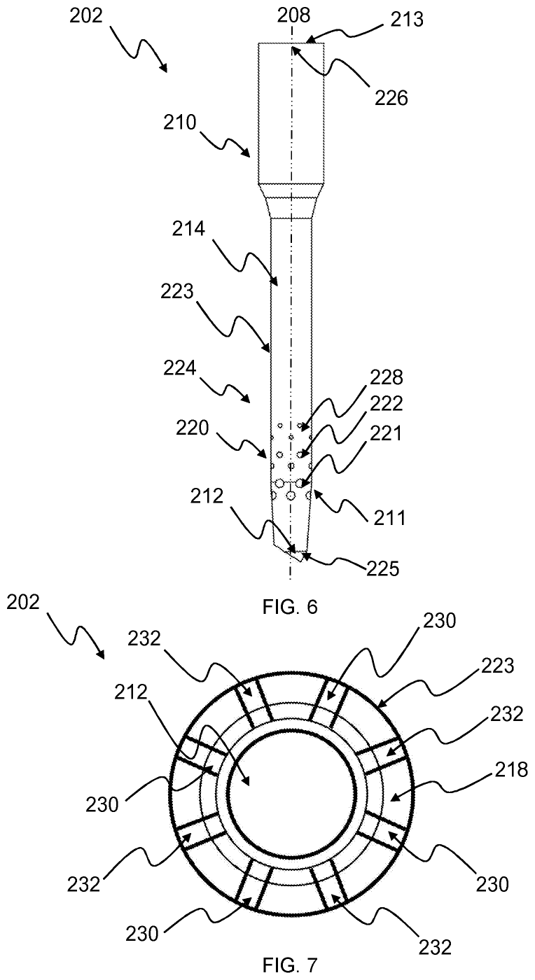

A cannula 202 according to an alternative embodiment of the present invention will now be described with particular reference to FIG. 6. The cannula 202 is applicable to the infusion device 1 according to some implementations. The cannula 202 is provided with a tubular body member 214 having a longitudinal extending internal bore or conduit 213 which extends along the longitudinal axis 208 of the cannula 202. A tubular wall 223 surrounds the bore 213. As described in relation to the cannula 2 of FIG. 2 and the cannula 102 of FIGS. 4 and 5, the conduit 213 interconnects the infusion device with an inlet opening 226 in the top and a tip opening 212. The cannula 202 includes a proximal portion 210, which is configured for interconnection with the infusion device (not shown in FIG. 6) as well as a distal portion 224 configured for subcutaneously placement by means of an insertion needle (not shown).

The outlet or tip opening 212 is arranged at the cannula tip or distal tip end 225 i.e., in the distal end 211 of the distal portion 224 of the cannula 202, opposite the interconnection with the infusion device. The distal tip end 225 may be shaped to a point, for example skived. The insertion impact of such a distal tip end 225 can beneficially be reduced in this way. The distal end may, in some embodiments of the invention, include a corrugated portion. The outlet 212 may or may not be considered the primary outlet for drugs. In addition to the outlet 212, the cannula 202 is provided with a weakened section 220. In this embodiment, the weakened section includes a first plurality of holes 221, a second plurality of holes 222 and a third plurality of holes 228. Each of the first plurality of holes 221, the second plurality of holes 222 and the third plurality of holes 228 are formed in the wall 226 at the distal end 211 of the cannula 202.

The first plurality of holes 221, which may include 4 to 16 holes, for example, are positioned closer to the distal end tip 225 than either of the second plurality of holes 222 or the third plurality of holes, each of which may include 4 to 16 holes. The third plurality of holes 228 are positioned further from the distal end tip 225 than either of the first plurality of holes 221 or the second plurality of holes 222. The second plurality of holes 222 are positioned intermediate the first plurality of holes 221 and the second plurality of holes 222.

In embodiments of the invention, the holes of the first plurality of holes 221 are at a distance in the range of 1.2 to 1.4 mm from the distal end tip 225. In embodiments of the invention, the holes of the first plurality of holes 221 are arranged in at least one row. Each row of the holes of the first plurality of holes may, for example, be equally spaced along the longitudinal axis 208 of the cannula 202. In embodiments of the invention, one row of the at least one row of the first plurality of holes 221 is at a distance of 1.2 mm from the distal end tip 225, a second row of the at least one row of the first plurality of holes 221 is at a distance of 1.3 mm from the distal end tip 225, and a third row of the at least one row of the first plurality of holes 221 is at a distance of 1.4 mm from the distal end tip 225.

In embodiments of the invention, the holes of the second plurality of holes 222 are at a distance in the range of 1.5 to 1.7 mm from the distal end tip 225. In embodiments of the invention, the holes of the second plurality of holes 222 are arranged in at least one row. Each row of the holes of the second plurality of holes 222 may, for example, be equally spaced along the longitudinal axis 208 of the cannula 202. In embodiments of the invention, one row of the at least one row of the second plurality of holes 222 is at a distance of 1.5 mm from the distal end tip 225, a second row of the at least one row of the second plurality of holes 222 is at a distance of 1.6 mm from the distal end tip 225, and a third row of the at least one row of the second plurality of holes 222 is at a distance of 1.7 mm from the distal end tip 225.

In embodiments of the invention, the holes of the third plurality of holes 228 are at a distance in the range of 1.8 to 2.0 mm from the distal end tip 225. In embodiments of the invention, the holes of the third plurality of holes 228 are arranged in at least one row. Each row of the holes of the third plurality of holes 228 may, for example, be equally spaced along the longitudinal axis 208 of the cannula 202. In embodiments of the invention, one row of the at least one row of the third plurality of holes 228 is at a distance of 1.8 mm from the distal end tip 225, a second row of the at least one row of the third plurality of holes 228 is at a distance of 1.9 mm from the distal end tip 225, and a third row of the at least one row of the third plurality of holes 228 is at a distance of 2.0 mm from the distal end tip 225.

In embodiments of the invention, each row of the first plurality of holes 221, the second plurality of holes 222 and the third plurality of holes 228 may include, for example, 4 holes. Each of the 4 holes in each row of the first plurality of holes 221, the second plurality of holes 222 and the third plurality of holes 228 may be equally spaced around the outer circumference of the tubular wall 223 such that each hole is at an angle of approximately 90° relative to its adjacent holes. Adjacent rows of the first plurality of holes 221, the second plurality of holes 222 and the third plurality of holes 228 may be offset, for example by 90°. The adjacent rows of the first plurality of holes 221 and the second plurality of holes 222 may also be offset, for example by 90°. Similarly, the adjacent rows of the second plurality of holes 222 and the third plurality of holes 228 may also be offset, for example by 90°. The arrangement of the first plurality of holes 221, the second plurality of holes 222 and the third plurality of holes 228 advantageously enhances the mechanical integrity of the cannula 202.

In embodiments of the invention, the first plurality of holes 221 include holes having a larger diameter than either of the holes of the second plurality of holes 222 or the holes of the third plurality of holes 228. The second plurality of holes 222 may also include holes having a larger diameter than the holes of the third plurality of holes 228. The holes of the first plurality of holes 221 may, for example, have a diameter in the range of 61 to 75 pm. The holes of the second plurality of holes 222 may, for example, have a diameter in the range of 56 to 60 pm. The holes of the third plurality of holes 228 may, for example, have a diameter in the range of 50 to 55 pm. The provision of holes having a larger diameter closer to the distal tip end 225 advantageously ensures that more of the drug is delivered closer to the distal tip end 225. Furthermore, in the event that kinking of the cannula occurs, such kinking is more likely to occur in the region of the first plurality of holes 221 and delivery of the drug will still be possible via the numerous holes of the second plurality of holes 222 and the third plurality of holes 228. In embodiments of the invention, the total opening area of the first plurality of holes 221, the second plurality of holes 222 and the third plurality of holes 228 may be approximately equal to the area of the tip opening 212.

The holes of each of the first plurality of holes 221, the second plurality of holes 222 and the third plurality of holes 228 are cut through the wall 223 allowing a fluid communication from the internal bore 213 to the outside of the cannula 202. A laser may cut the holes of each of the first plurality of holes 221, the second plurality of holes 222 and the third plurality of holes 228. Each hole of each of the first plurality of holes 221, the second plurality of holes 222 and the third plurality of holes 228 has an opposing, or mirror, hole on the opposite side of the tubular wall 223. This advantageously reduces the time taken for the holes to be cut into the cannula 202.

When the cannula 202 is subjected to a compression force or an increased internal pressure such that the internal pressure in the longitudinal extending bore 213 exceeds the internal pressure at the tip opening 212, the weakened section 220 provides a fluid communication between the internal bore 213 and the outside of the cannula 202.

Referring now to FIG. 7, a cross section view of the cannula 202 is shown. Four holes 230 of one row of the first plurality of holes 221, second plurality of holes 222 or third plurality of holes 228 and four holes 232 of an adjacent row of the first plurality of holes 221, second plurality of holes 222 or third plurality of holes 228 can be seen, along with the eight slats 218 which are formed between them.

In use, pressure may arise from the tip 125, 225 of the cannula 102, 202 contacting a hard element. Instead of kinking, and thereby causing an obstruction, the weakened section 120, 220 I the first plurality of holes 121, 221, second plurality of holes 122, 222, the third plurality of holes 228 and the slats 218 provide a flexible portion of the distal end 111, 211 of the cannula 102, 202 in the area where the weakened section 120, 220 I the first plurality of holes 121, 221, second plurality of holes 122, 222, the third plurality of holes 228 and the slats 218 are positioned. The slats are bending outwards thereby providing several openings in the wall of the cannula allowing the drug to flow from the inside of the cannula 102, 202 through the first plurality of holes 121, 221, second plurality of holes 122, 222, and the third plurality of holes 228 and into the subcutaneous tissue. The pressure may also arise from internal pressure in the longitudinal extending internal bore 113, 213. This may be caused by clogging of the tip opening 112, 212 of the cannula 102, 202 thereby preventing drug delivery through the tip opening 112, 212. Due to the increased pressure, the slats 218 will bend outward away from the internal bore 113, 213, and the first plurality of holes 121, 221, second plurality of holes 122, 222, and the third plurality of holes 228 will open wider providing a larger fluid path from the inside of the cannula 102, 202 to the subcutaneous tissue. When the pressure normalizes the first plurality of holes 121, 221, second plurality of holes 122, 222, and the third plurality of holes 228 will return to their original size, and the drug will preferentially leave the cannula 102, 202 through the tip opening 112, 212 and optionally through the first plurality of holes 121, 221, second plurality of holes 122, 222, and the third plurality of holes 228.

In the event that a small part of the tip contacts a hard element and thereby the compression force is only compressing the cannula on that side of the wall 123, 223 where the element is located, the slats 218 become most deformed in this area: that is they are bending outwards whereby the first plurality of holes 121, 221, second plurality of holes 122, 222, and the third plurality of holes 228 are opened wider and provide larger openings on that side of the cannula 102, 202 where the compression of the cannula 102, 202 takes place.

In some implementations, a laser beam is used to cut the first plurality of holes 121, 221, second plurality of holes 122, 222, and the third plurality of holes 228 through the cannula 102, 202. In general, the diameter of the first plurality of holes 121, 221, second plurality of holes 122, 222, and the third plurality of holes 228 may be based on the laser equipment used to form the slits. Further, in some instances, a single laser beam can form two slits substantially simultaneously, e.g., two slits that are separated by about 180° from one another. In some instances, the first plurality of holes 121, 221, second plurality of holes 122, 222, and the third plurality of holes 228 are cut using a femtosecond laser. In some instances, the first plurality of holes 121, 221, second plurality of holes 122, 222, and the third plurality of holes 228 are cut using a femtosecond laser where a hole of the first plurality of holes 121, 221, second plurality of holes 122, 222, or the third plurality of holes 228 and an opposing hole of the first plurality of holes 121, 221, second plurality of holes 122, 222, and the third plurality of holes 228 are cut in the same instance.

In some embodiments the distal portion and/or the distal end of the cannula 102, 202, or in some cases the entire cannula 102, 202, can be composed, for example, of a soft material such as PTFE (polytetrafluoroethylene; Teflon™), FEP (fluorinated ethylene propylene), rubber, PE material or silicone base materials and the like.

As described above, soft cannulas can be impacted negatively by contact with tissue, which can cause bending or kinking of the cannula. Such bending or kinking can result in a decreased or interrupted flow of the therapeutic agent. When the cannula 102, 202 is not exposed to kinking or occlusion of the tip opening 112, 212, the therapeutic agent can leave the cannula bore through the tip 112, 212 and/or through the first plurality of holes 121, 221, second plurality of holes 122, 222, and/or the third plurality of holes 228.

On the other hand, the tip opening 112, 212 may become obstructed or kinking may occur, for example by the tip opening 112, 212 of the cannula 102, 202 coming into straight or perpendicular contact with fascia or other tissue. The cannula 102, 202 is adapted to flex in an area comprising the first plurality of holes 121, 221 when the cannula is exposed to a significant compression force and/or an increased internal pressure inside the cannula longitudinal extending bore. Thus, if the tip opening 112, 212 is obstructed, the pressure inside the cannula 102, 202 increases and the weakened section comprising the first plurality of holes 121, 221, the second plurality of holes 122, 222, and/or the third plurality of holes 228 flex outward, thereby providing openings in the wall to allow the therapeutic agent to leave through the openings provided by the first plurality of holes 121, 221, second plurality of holes 122, 222, and/or the third plurality of holes 228. Accordingly, even if the tip opening 112, 212 is obstructed, one or more of the holes of the first plurality of holes 121, 221, second plurality of holes 122, 222, and/or the third plurality of holes 228 can provide fluid communication between the internal bore and the outside of the cannula.

In other scenarios, the tip opening 112, 212 of the cannula 102, 202 may come into contact at an angle with fascia or other tissue. If kinking occurs in the cannula 102, 202, the kinking will tend to occur at areas comprising one or more of the larger holes of the first plurality of holes 121, 221, thereby allowing the therapeutic agent to be delivered through one or more other holes (e.g., slits on the opposite side of the cannula from where the kinking occurs and/or holes of the second plurality of holes 122, 222 and/or the third plurality of holes 228).

In some embodiments, at least one of the first plurality of holes 121, 221, second plurality of holes 122, 222, and/or the third plurality of holes 228 provides an opening between the internal bore and the outside of the cannula, wherein the therapeutic agent (e.g., a drug) may leave the inside of the cannula even in the presence of an obstruction or occlusion. For example, if kinking occurs in a cannula, such kinking may occur within the first plurality of holes 121, 221 of the cannula, allowing the therapeutic agent (e.g., a drug) to be delivered through the second plurality of holes 122, 222, and/or the third plurality of holes 228. Likewise, if the distal tip of the outlet is obstructed, the pressure inside the cannula increases and the weakened section comprising the first plurality of holes 121, 221, second plurality of holes 122, 222, and the third plurality of holes 228 will flex outwards providing larger openings in the wall and through the openings, the fluid will leave.

In some embodiments, of the infusion devices and cannulas disclosed herein, even when the cannula does not kink or the internal pressure does not exceed the pressure at the tip of the cannula, at least one of the first plurality of holes 121, 221, second plurality of holes 122, 222, and/or the third plurality of holes 228 provides a fluid communication between the internal bore of the cannula and the outside of the cannula.

“Same circumferential location” indicates that the holes of the first plurality of holes 121, 221, the second plurality of holes 122, 222 and/or the third plurality of holes 228 are placed in such a way that the upper end of a hole is at the same level as the upper end of the first neighbouring hole or between the upper end and the lower end of the first neighbouring hole, and the lower end of the hole is at the same level as the lower end of the second neighbouring hole or between the upper end and the lower end of the second neighbouring hole. By “upper” is meant closest to the proximal portion of the cannula, and by “lower” is meant closest to the distal end or tip of the cannula.

In some embodiments, the weakened section is formed as a plurality of holes and formed at the same circumferential location of the tubular body member, the plurality of holes providing slats placed between the plurality of holes, the slats are adapted to flex outwards away from the internal bore when the cannula is exposed by compression forces and/or an increased internal pressure. In some embodiments, the slats are delimited by sidewalls parallel to the longitudinal axes of the tubular body member and the sidewalls delimiting each slat are parallel to each other in a radial direction. In some embodiments, the slats are delimited by sidewalls parallel to the longitudinal axis of the tubular body in that the sidewalls for each slat converge in a radial direction towards the outside of the tubular body member, wherein the openings provided by the plurality of holes are converging towards the internal bore.

According to some implementations, the weakened section is are formed as a first plurality of holes, a second plurality of holes and/or a third plurality of holes and formed at the same circumferential location of the tubular body member, the first plurality of holes, a second plurality of holes and/or a third plurality of holes providing slats between the first plurality of holes, a second plurality of holes and/or a third plurality of holes, the slats adapted to flex outwards away from the internal bore when the cannula is exposed to compression forces or when the internal pressure exceeds the pressure at the tip opening. In some embodiments, the bending of the slats may take place substantially in the middle of the slats.

In some embodiments, the first plurality of holes, a second plurality of holes and/or a third plurality of holes are formed by laser cutting of a set portion of the tubular wall. In some instances, no residuals are left behind when laser cutting is used to form the slits in the tubular wall of the devices disclosed herein. In some instances, no cleaning of the first plurality of holes, a second plurality of holes and/or a third plurality of holes is needed to form the first plurality of holes, a second plurality of holes and/or a third plurality of holes of the devices disclosed herein.

In some embodiments, the weakened section comprises a part of the wall of the tubular body member of the cannula being formed in a material having a smaller compression strength than the rest of the wall of the tubular body member. In some embodiments, the weakened section comprises a plastic, including but not limited to PTFE (polytetrafluoroethylene), rubber or PE (polyethylene). In still other embodiments, the first weakened section comprises a part of the wall of the tubular body member having a thickness that is thinner than the surrounding wall of the tubular body member, thereby having a smaller compression strength than the rest of the wall of the tubular member.

In some embodiments, the weakened section comprises a part of the wall of the tubular body member being formed that corrugated in the weakened portion region and shaped into alternate ridges and grooves.

In some embodiments, the infusion device further comprises a pump in fluid connection with a reservoir configured to store medication or other therapeutic drug or agent.

In some embodiments, the subcutaneously placed distal portion of the cannula comprises a soft material such as PTFE (polytetrafluoroethylene; Teflon), FEP (fluorinated ethylene propylene), rubber, PE (polyethylene) material or silicone base materials. According to some implementations, the cannula is insertable with an insertion needle. According to some implementations, a length of the distal end of the cannula is less than 3.5 mm, less than 3.25 mm, less than 3.0 mm, less than 2.75 mm, less than 2.5 mm, less than 2.0 mm, less than 1.75 mm, less than 1.5 mm, less than 1.25 mm, or less than 1.5 mm. In some embodiments, a length of the distal end is less than 2.5 mm, and an outer diameter of the distal end is less than 1.5 mm.

In some embodiments, the infusion device is configured for subcutaneous infusion of one or more drugs or other therapeutic agents. According to some implementations, the one or more therapeutic agents comprise insulin.

According to some implementations, the weakened section is positioned below the basal membrane when the cannula is subcutaneously placed.

In some embodiments, the aspects of the present disclosure include a method of administering a therapeutic agent via an infusion device providing a cannula having a tubular body member comprising a tubular wall at least partly enclosing a longitudinal extending internal bore, a distal portion of the tubular body member having a distal tip end comprising at least one tip opening, wherein the tubular wall comprises a weakened section comprising a first plurality of holes and a second plurality of holes, the first plurality of holes closer to the distal end tip than the second plurality of holes, and wherein the first plurality of holes comprise holes of larger diameter than the second plurality of holes; and causing the cannula to flex in an area comprising the weakened section when 1) the cannula is exposed to a compression force whereby the tip opening is substantially closed and/or 2) the cannula is exposed to an increased internal pressure exceeding the pressure at the tip opening, wherein the compression force or increased internal pressure opens the weakened section allowing fluid communication between the internal bore and the external environment, thereby discharging the therapeutic agent.

In some embodiments, an infusion device comprises: a cannula comprising a tubular body member comprising a tubular wall at least partly enclosing a longitudinal extending internal bore, a distal portion of the tubular body member having a distal tip end comprising at least one tip opening, wherein the distal portion comprises a weakened section comprising a first plurality of holes and a second plurality of holes, the first plurality of holes closer to the distal end tip than the second plurality of holes, the weakened section being capable of allowing the cannula to flex in an area comprising the weakened section when the cannula is exposed to a compression force and/or an increased internal pressure, wherein the first plurality of holes comprise holes of larger diameter than the second plurality of holes; and a hub part configured to be fastened onto the patient's skin via a mounting pad.

According to some implementations, the bending of the slats takes place approximately in the middle of the holes of the first plurality of holes, a second plurality of holes and/or a third plurality of holes.

Other implementations are within the scope of the claims. As used herein, as one of ordinary skill in the art would readily appreciate, the examples and embodiments described herein in connection with to a “therapeutic agent,” “drug” or “fluid” are equally applicable to therapeutic agents, fluids, drugs, suspensions and other conventional materials suitable for delivery via a cannula.

The specification and drawings are to be regarded in an illustrative rather than a restrictive sense. It will, however, be evident that various modifications and changes may be made thereunto without departing from the broader spirit and scope of the invention as set forth in the claims. Based on the disclosure and teachings provided herein, a person of ordinary skill in the art will appreciate other ways and/or methods to implement the various embodiments.

1. A cannula for subcutaneous infusion of a therapeutic agent, the cannula comprising a tubular body member comprising a tubular wall at least partly enclosing a longitudinal extending internal bore, a distal portion of the tubular body member having a distal tip end, wherein the distal portion comprises a weakened section comprising a first plurality of holes and a second plurality of holes, the weakened section being capable of allowing the cannula to flex in an area comprising the weakened section when the cannula is exposed to a compression force and/or an increased internal pressure, and wherein the first plurality of holes comprise holes of larger diameter than the second plurality of holes.

2. The cannula of claim 1, wherein the first plurality of holes are closer to the distal tip end than the second plurality of holes.

3. The cannula of claims 1 or 2, wherein the number of holes in the first plurality of holes is between 4 to 16.

4. The cannula of any of the preceding claims, wherein the number of holes in the second plurality of holes is between 4 to 16.

5. The cannula of any of the preceding claims, wherein the first plurality of holes have a diameter in the range of 61 to 75 pm.

6. The cannula of any of the preceding claims, wherein the second plurality of holes have a diameter in the range of 50 to 60 pm.

7. The cannula of any of the preceding claims, wherein the distal tip end is shaped to a point.

8. The cannula of any of the preceding claims, wherein the cannula is arranged so that at least a portion of the therapeutic agent is released from the distal tip end.

9. The cannula of any of the preceding claims, wherein the weakened section has a compression strength smaller than a compression strength of remaining portions of the tubular body member.

10. The cannula of any of the preceding claims, wherein when the cannula is exposed to a compression force and/or an increased internal pressure and an internal pressure
in the longitudinal extending bore exceeds an internal pressure in a tip opening of the distal tip end, the weakened section provides a fluid communication between the internal bore and outside of the cannula.

11. The cannula of any of the preceding claims, wherein each of the first plurality of holes are at the same distance from a tip opening of the distal tip end.

12. The cannula of claim 1, wherein the distal tip end further comprises a corrugated portion.

13. The cannula of claim 1, wherein the distal portion of the cannula comprises PTFE (polytetrafluoroethylene), FEP (fluorinated ethylene propylene), rubber, PE (polyethylene) material or silicone base materials.

14. The cannula of claim 1, wherein the cannula is configured to be insertable with an insertion needle.

15. The cannula of claim 1, wherein the cannula is configured such that the weakened section is below a basal membrane of a patient's skin when the cannula is subcutaneously placed.

16. The cannula of claim 1, wherein a length of the distal tip end of said cannula is less than 3.5 mm, and an outer diameter of the distal tip end is less than 1.5 mm.

17. An infusion device for subcutaneous delivery of a therapeutic agent to a patient comprising:
a cannula comprising a tubular body member comprising a tubular wall at least partly enclosing a longitudinal extending internal bore, a distal portion of the tubular body member having a distal tip end, wherein the distal portion comprises a weakened section comprising a first plurality of holes and a second plurality of holes, the weakened section being capable of allowing the cannula to flex in an area comprising the weakened section when the cannula is exposed to a compression force and/or an increased internal pressure, wherein the first plurality of holes comprise holes of larger diameter than the second plurality of holes; and
a hub part configured to be fastened onto a patient's skin via a mounting pad.

18. The infusion device of claim 17, wherein the first plurality of holes are closer to the distal tip end than the second plurality of holes

19. The infusion device of claim 17, wherein the device is configured such that at least a portion of the therapeutic agent is released from the distal tip end of the cannula.

20. The infusion device of claim 17, wherein the weakened section has a compression strength smaller than a compression strength of remaining portions of the tubular body member.

21. The infusion device of claim 17, wherein when the cannula is exposed to a compression force and/or an increased internal pressure and an internal pressure in the longitudinal extending bore exceeds an internal pressure in a tip opening of the distal tip end, the weakened section provides a fluid communication between the internal bore and outside of the cannula.

22. The infusion device of claim 17, wherein the distal tip end of the cannula comprises a corrugated portion.

23. The infusion device of claim 17, wherein the distal portion of the cannula comprises PTFE (polytetrafluoroethylene), FEP (fluorinated ethylene propylene), rubber, PE (polyethylene) material or silicone base materials.

24. The infusion device of claim 17, wherein the cannula is configured to be insertable with an insertion needle.

25. The infusion device of claim 17, wherein the cannula is configured such that the weakened section is positioned below a basal membrane of the patient's skin when the cannula is subcutaneously placed.

26. The infusion device of claim 17, wherein a length of the distal tip end of said cannula is less than 3.5 mm, and an outer diameter of the distal tip end is less than 1.5 mm.

27. The infusion device of claim 17, wherein the diameter of the first plurality of holes is in the range of 61 to 75 pm.

28. The infusion device of claim 17, wherein the device further comprises a pump in fluid connection with a reservoir configured to store medication/drugs.

29. The infusion device of claim 17, wherein the subcutaneously placed distal portion of said cannula comprises a soft material such as PTFE (polytetrafluoroethylene), FEP (fluorinated ethylene propylene), rubber, PE (polyethylene) material or silicone base materials.

30. The infusion device of claim 17, wherein said infusion device is configured for subcutaneous infusion of one or more therapeutic agents.

31. The infusion device of claim 17, wherein the therapeutic agent comprises insulin.

32. A method of administering a therapeutic agent via an infusion device, the method comprising:
providing a cannula comprising a tubular body member comprising a tubular wall at least partly enclosing a longitudinal extending internal bore, a distal portion of the tubular body member having a distal tip end comprising at least one tip opening, wherein the tubular wall comprises a weakened section comprising a first plurality of holes and a second plurality of holes, and wherein the first plurality of holes comprise holes of larger diameter than the second plurality of holes; and
causing the cannula to flex in an area comprising the weakened section when 1) the cannula is exposed to a compression force whereby the tip opening is substantially closed and/or 2) the cannula is exposed to an increased internal pressure exceeding the pressure at the tip opening, wherein the compression force or increased internal pressure opens the weakened section allowing fluid communication between the internal bore and the external environment, thereby discharging the therapeutic agent.

33. The method of claim 32, wherein the first plurality of holes are closer to the distal tip end than the second plurality of holes.
